(19) United States
(12) Patent Application Publication
(10) Pub. No.: US 2009/0162888 A1
(43) Pub. Date: Jun. 25, 2009

(54) SAMPLE CONTROL FOR CORRECTION OF SAMPLE MATRIX EFFECTS IN ANALYTICAL DETECTION METHODS

(75) Inventors: Kristiane Anne Schmidt, Eindhoven (NL); Gerardus Wilhelmus Lucassen, Eindhoven (NL); Sieglinde Neerken, Eindhoven (NL)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., EINDHOVEN (NL)

(21) Appl. No.: 12/300,519

(22) PCT Filed: May 8, 2007
(86) PCT No.: PCT/IB07/51725

§ 371 Date: Nov. 12, 2008

(30) Foreign Application Priority Data
May 16, 2006 (EP) 06114018.2

(51) Int. Cl. C12Q 1/02; G01N 21/00; G01N 33/00; G01N 33/68

(52) U.S. Cl. 435/29; 436/164; 436/94; 436/86; 436/71; 422/82.05; 422/55

(57) ABSTRACT

Methods and systems are described suitable to determine the effects of sample matrix on the detection of a label so as to allow correction for these sample matrix effects when using the label in an analytical detection technique. The method is particularly advantageous for use in a disposable molecular diagnosis cartridge.

The present invention relates to a method for determining the occurrence of sample matrix effects on the detection of a label which allows for the correction of these sample matrix effects in an analytical technique involving the use of this or a similar label as well as to devices operating in accordance with the method.

The sensitive and accurate detection, either qualitatively or quantitatively, of biomolecules such as proteins, peptides, oligonucleotides, nucleic acids, lipids, polysaccharides, hormones, neurotransmitters, metabolites, etc. has proven to be an elusive goal despite widespread potential uses in medical diagnostics, pathology, toxicology, epidemiology, biological warfare, environmental sampling, forensics and numerous other fields such as comparative proteomics and gene expression studies.

Particular examples relating to the detection of DNA are, e.g. in medical diagnostics for example the detection of infectious agents like pathogenic bacteria and viruses, the diagnosis of inherited and acquired genetic diseases, etc., in forensic tests as part of criminal investigations, in paternity disputes, in whole genome sequencing, etc.

While the identification and/or quantification of a purified sample of a biological analyte can sometimes be performed based on the physicochemical properties of the analyte itself, most detection methods which are capable of identifying and/or quantifying an analyte in a non-purified sample make use of a “probe” which is a known molecule having a strong affinity and preferably also a high degree of specificity for the analyte. Where the analyte is a protein or peptide, these assays are referred to as ligand-binding assays (e.g. immunoassays). Detection of DNA typically makes use of the hybridization of a nucleotide sequence which is specific for the target DNA.

In these probe-based detection assays the analyte-specific probe (or the analyte) is either directly or indirectly labeled with a traceable substance. The detection of the traceable substance (hereafter referred to as “label”) bound via the probe to the analyte, is indicative of the amount of analyte in the test sample. Detection of the label can be ensured using a variety of different techniques, depending upon the nature of the label employed used.

One biotechnological analytical technique is Raman spectroscopy. In Raman spectroscopy the inelastic scattering of light (called Raman scattering) by molecules in a sample is detected. The resulting Raman spectrum is characteristic of the chemical composition and structure of the light absorbing molecules in the sample, while the intensity of the Raman scattering is dependent on the concentration of these molecules.

The observation that emission spectra are enhanced by several orders of magnitude, up to 10¹⁴-fold, when molecules are adsorbed onto roughened metal surfaces, e.g. nanoparticles of gold, silver, copper and certain other metals, has resulted in highly sensitive surface-enhanced spectroscopies (e.g. surface-enhanced fluorescence (SEF) and surface-enhanced (resonance) Raman spectroscopy (SE(R)RS)).

In surface-enhanced Raman resonance spectroscopy (SERRS), use is made of a “SERRS-active” substance or dye attached to the analyte (capable of generating a SERRS spectrum when appropriately illuminated), and operating at the resonance frequency of the dye.

Critical steps in the use of surface-enhanced spectroscopies are the reproducible production of roughened metal surfaces and the efficient adsorption/binding of the label to be detected onto this metal surface. When the roughened metal surface consists of colloidal metal nanoparticles the best signal enhancement is achieved when they are aggregated in a controlled manner. Unaggregated colloids are prepared by, for instance, the reduction of a metal salt (e.g. silver nitrate) with a reducing agent such as citrate, to form a stable microcrystalline suspension. This colloidal suspension is then aggregated immediately prior to use. Ideally the aggregated colloids are formed in situ in the sample and the SE(R)RS spectrum is obtained shortly afterwards so as to prevent precipitation.

It has been observed that in any detection technique making use of a label which requires detection within the sample, sample matrix effects can influence the results of an analysis. Sample matrix effects are especially severe in complex media such as biological, mineralogical, or environmental samples where the nature and amounts of interfering substances are often unknown and not readily controlled, but can also be relevant in samples which are obtained from (semi-)purification techniques, due to the presence of salts and/or other components which can influence different aspects of the detection. In biological samples the sample matrix effect can be caused by an excess of bodily fluid constituents such as lipemia, bilirubinemia, hemoglobinemia, hemolysis, lipids, proteins, hemoglobin, immunoglobin, hormones, drugs, antigens, allergens, toxins, tumor markers, soluble cell molecules, and nucleic acid. In DNA extracts, the sample matrix effect can be caused by the mere presence of bulk DNA. These constituents may either increase or decrease the measurement signal, causing an inaccurate result. Sample matrix effects can be manifested e.g. by quenching of fluorescence or luminescence.

Surface-enhanced spectroscopies provide an additional complexity in that the sample matrix can interfere with the colloid aggregation as well as with the adsorption/binding of the analyte or label onto the colloid. Different degrees of aggregation of metal colloids result in a variable signal. These variations in colloid aggregation can be caused by differences in pH of samples or by the presence of ions that induce over-aggregation resulting in precipitation of the aggregates. Sample matrix compounds may also adsorb onto the metal particles thereby competing for the surface of the nanoparticle with the molecule of which the signal is to be enhanced. For example, many proteins that tend to be positively charged at neutral or physiological pH are attracted to the net negative charge of the particles. Antibodies especially tend to adsorb strongly to colloid gold particles. Sample matrix compounds could also contribute to non-specific adsorption of label to nanoparticles.

Correcting for factors affecting detection is a general problem in the field of analytical chemistry. In the art, approaches to eliminate sample matrix effects include dilution, removal of the sample matrix (e.g. by covalently binding the analyte to a fixed surface and washing the background off without affecting the analyte), and the addition of a standard and subsequent correction for the degree of interference.

Methods compensating for sample matrix effects have been developed for SE(R)RS methods based on the direct detection of the sample matrix effects in the sample. These methods involve the use of an internal standard which is a predetermined amount of a molecule which is comparable to the molecule to be detected (e.g. analyte or label), but generates a different signal. These techniques are, however, limited by the fact that the effects are measured on a compound which is not the same, and thus that the spectroscopic signalling efficiencies and the interference of sample matrix with the detection could be different.

An object of the present invention is to provide an alternative method for correcting the sample matrix effects in analytical techniques as well as systems operating in accordance with the method. An advantage of the present invention is that the negative effects on the detection of the label in the sample are reduced by a label control that permits the determination of sample matrix effects.

In a first aspect, the invention provides methods for determining sample matrix effects of a sample on the detection of a label, the method comprising the steps of (a) contacting a predetermined amount of the same label or a different label, with a background sample comprising sample matrix or sample-like matrix; (b) contacting a predetermined amount of the same label, or of the different label, with a background-free sample not comprising sample matrix or sample-like matrix or any other compound which is capable of interfering with the detection of the label; (c) detecting the same or different label in the background sample and the background-free sample; and (d) determining a difference between the detection of the same or different label in the background sample and the background-free sample, thereby obtaining the sample matrix effects. In a preferred embodiment all the predetermined amounts are the same which makes the correction easier to perform, only involving simple differences.

A second aspect of the invention provides methods for determining sample matrix effects on the detection of an analyte in a sample, the method comprising the steps of (a) providing a test sample from the sample in which the analyte is to be detected, a background sample comprising sample matrix or sample-like matrix, and a background-free sample, not comprising sample matrix or sample-like matrix; (b) detecting and/or quantifying the analyte in the test sample using a label; (c) detecting the sample matrix effects, by a method comprising the steps of

contacting the background sample with a predetermined amount label, which can be the same or a different label,

contacting the background-free sample with a predetermined amount of the same or different label, whereby the same label is added to both the background and the background-free sample

detecting the same or different label in the background sample and in the background-free sample,

determining the sample matrix effects by determining a difference between the detection of the (same or different) label in the background sample and the background-free sample; and

(d) correcting the detection and/or quantification of the analyte in the test sample as obtained in step (b) with the sample matrix effects determined as described above.

In a preferred embodiment all the predetermined amounts are the same which makes the correction easier to perform, only involving simple differences.

Typically, the detection of the analyte in the test sample is performed by the detection of a label capable of binding to the analyte and the correction is performed by correcting the detection value of this bound label with the value obtained for the sample matrix effects.

According to one embodiment of this aspect of the invention, the background sample is a fraction of a sample in which an analyte is to be detected. Additionally or alternatively, the background sample comprises sample matrix or sample-like matrix.

The analytes which are envisaged to be detected by the methods of the present invention are, in one embodiment selected from the group consisting of a nucleic acid, a protein, a carbohydrate, a lipid, a chemical substance, an antibody, a microorganism, and a eukaryotic cell.

According to one embodiment, the detection step in the methods of the present invention, is performed using an optical detection method. Most particularly, the detection method is SE(R)RS, and the label used in both the determination of the sample matrix effects and the detection and/or quantification of the analyte is a SE(R)RS-active label. Typically, such methods involve an additional step whereby, prior to detection of the label in the different samples, the label is contacted with a SE(R)RS-active surface. According to a particular embodiment, the SE(R)RS-active surface is a colloidal suspension of silver or gold nanoparticles, or aggregated colloids thereof.

According to one embodiment, in the methods involving the detection and/or quantification of an analyte, this detection and/or quantification is performed using a labeled analyte-specific probe. According to a particular embodiment, the analyte-specific probe is provided with a binding-sensitive label, i.e. a label of which the detection signal is modified upon binding to the analyte.

According to one embodiment, the analyte to be detected is a nucleotide sequence and use is made of a labeled analyte-specific probe which is a nucleotide or nucleotide having a sequence complementary to a sequence within the analyte.

Detection of the analyte using a labeled analyte-specific probe can be based on the direct detection of labeled analyte-specific probe bound to the analyte or can be based on a competitive binding of the analyte. According to a specific embodiment of this latter embodiment, the detection of the analyte is performed using an analyte-specific probe capable of binding to a SE(R)RS-active surface and a labeled surrogate probe, and whereby the analyte competes with the labeled surrogate probe for the binding to the analyte-specific probe.

Yet another aspect of the present invention provides devices or systems for compensating for sample matrix effects on the detection of an analyte or a label in a sample.

The present invention provides a system for determining sample matrix effects of a sample on the detection of a label, comprising:

(a) means for contacting a predetermined amount of said label or a different label, with a background sample comprising sample matrix or sample-like matrix,
(b) means for contacting a predetermined amount of said label, or of said different label, with a background-free sample not comprising sample matrix or sample-like matrix or any other compound which is capable of interfering with the detection of the label,
(c) means for detecting said label or said different label in said background sample and said background-free sample, and
(d) means for determining a difference between the detection of said label or said different label in said background sample and in said background-free sample, corresponding to said sample matrix effects.

The present invention also provides a system for determining sample matrix effects on the detection of an analyte in a sample comprising:

(a) means for providing a test sample from said sample in which said analyte is to be detected, a background sample comprising sample matrix or sample-like matrix, and a background-free sample, not comprising sample matrix or sample-like matrix,
(b) means for detecting and/or quantifying the analyte in said test sample using a label,
(c) means for contacting said background sample with a predetermined amount of said label or a different label,
(d) means for contacting said background-free sample with a predetermined amount of said label or said different label,
(e) means for detecting said label or said different label in said background sample and in said background-free sample,
(f) means for determining said sample matrix effects by determining a difference between the detection of said label or said different label in said background sample and said background-free sample,
(g) means for correcting the detection and/or quantification of said analyte in said test sample responsive to the means for determining the sample matrix effects.

In a preferred embodiment all the predetermined amounts are the same which makes the correction easier to perform, only involving simple differences.

The system may comprise:

a first source of one or more samples selected from the group consisting of test sample containing the analyte, background sample and background-free sample, a second source of one or more labels and optionally a third source of additives,

means for providing the samples, labels and additives of the first to third sources so that they can be contacted.

According to a specific embodiment, the first source (101, FIG. 3) comprises chambers for the test sample containing analyte, the background sample and the background-free sample, respectively. The means for contacting can comprise chambers for contacting the test sample containing the analyte, background sample and background-free sample, respectively with the relevant labels.

In a further specific embodiment, the second source (102, FIG. 3) includes a chamber for an analyte-specific label and a chamber for at least one label.

The present invention also provides a disposable cartridge for use in a system for determining sample matrix effects on the detection of an analyte in a sample, comprising:

a first source of one or more samples selected from the group consisting of the test sample containing the analyte, background sample and background-free sample, and a second source of one or more labels and optionally a third source of additives, and

means for contacting the background sample with a predetermined amount of the same or a different label, for contacting the background-free sample with a predetermined amount of the same or a different label, and for contacting the test sample with a predetermined amount of the same or a different label, and

a window to allow detection of that same or a different label in the test sample, the background sample and the background-free sample. The source of the sample to be tested can be a PCR reaction chamber.

The above and other characteristics, features and advantages of the present invention will become apparent from the following detailed description, taken in conjunction with the accompanying drawings, which illustrate, by way of example, the principles of the invention. This description is given for the sake of example only, without limiting the scope of the invention.

The present invention will now be described with reference to the following drawings.

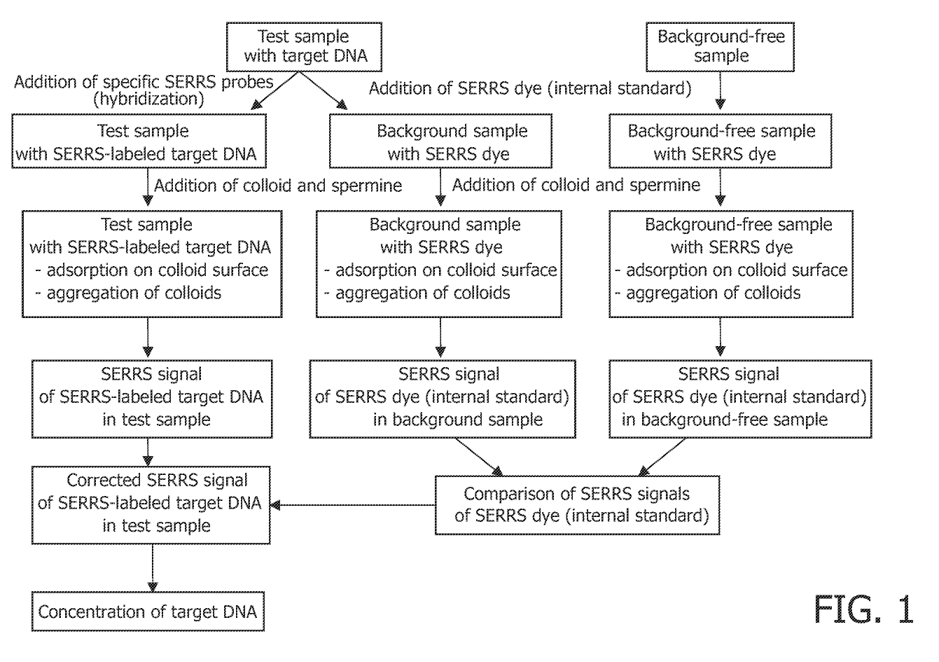

FIG. 1 is a schematic drawing of an embodiment of the method of the present invention to detect an analyte in a test sample including the detection of the label control by detecting the label in a background matrix and in background-free matrix, as applied to SE(R)RS detection of DNA in a test sample.

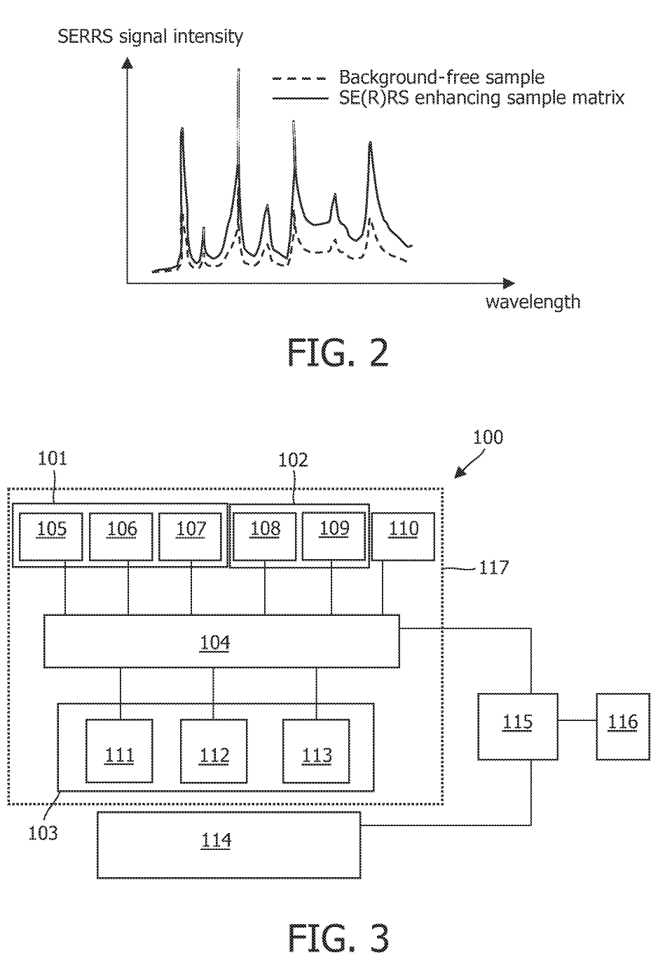

FIG. 2 is an example of SERRS spectra of a SERRS-active label in a background-free sample (1) and SERRS-active label in a sample matrix that enhances the SERRS effect (2) according to one embodiment of the invention. A comparison of these two spectra gives information on the sample matrix effects that possibly interfere with the analyte detection in the test sample.

FIG. 3 is a schematic representation of the system according to an embodiment of the present invention.

The present invention will be described with respect to particular embodiments and with reference to certain drawings but the invention is not limited thereto but only by the claims. Any reference signs in the claims shall not be construed as limiting the scope. The drawings described are only schematic and are non-limiting. In the drawings, the size of some of the elements may be exaggerated and not drawn on scale for illustrative purposes. Where the term “comprising” is used in the present description and claims, it does not exclude other elements or steps. Where an indefinite or definite article is used when referring to a singular noun e.g. “a” or “an”, “the”, this includes a plural of that noun unless something else is specifically stated.

Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein.

The following terms or definitions are provided solely to aid in the understanding of the invention. These definitions should not be construed to have a scope less than understood by a person of ordinary skill in the art.

The term “analyte”, as used herein, refers to the substance to be detected and/or quantified in the methods of the present invention.

The term “label”, as used herein, refers to a molecule or material capable of generating a detectable signal. Unless specified this refers to the molecule as such, not covalently linked to a probe. The label could be attached to a probe, attached to an analyte or it could be a separate entity which binds to an analyte and/or a probe. An “analyte-specific probe” as used herein, is a probe comprising a structure or sequence which is specific for the analyte to be detected. This includes both compounds which are capable of specifically binding to the analyte (“complementary target probe”), and compounds which are at least similar to a specific part of the analyte (“surrogate target probe”). The binding of the complementary target probe to the analyte can be based on any type of interaction including but not limited to complementary nucleotide sequences, antigen/antibody interaction, ligand/receptor binding, enzyme/substrate interaction, etc. The surrogate target probe is used in competitive assays where the analyte is determined based on competition with the surrogate target probe, e.g. in the competitive binding to an analyte-specific probe. Most particularly, the surrogate target probe binds to an analyte-specific probe with a reduced binding strength compared to the binding of the analyte to the analyte-specific probe.

A “(SE(R)RS-active) surface” as used herein refers to a material which is capable of enhancing the signal of a SE(R)RS-active label.

A “capture probe” as used herein refers to a molecule capable of binding a molecule or a complex of molecules to a substrate. An analyte-specific capture probe, is a probe capable of specifically binding an analyte to a substrate.

A “substrate” as used herein refers to a material, to which molecules or complexes of molecules can be bound, either directly or by way of a capture probe, and which can be manipulated. Typical examples of substrates include but are not limited to microtiter plates, beads, chips, etc.

The term “sample” as used herein as such relates to a composition comprising a matrix (“sample matrix”) and therein the analyte of interest.

The term “test sample” is understood to mean a sample, or a fraction thereof, comprising a matrix (“sample matrix”) and therein the analyte of interest on which detection of the analyte is performed.

The term “sample matrix” is understood to mean the compounds present in the test sample which are not the analyte.

The term “sample-like matrix” is understood to mean a matrix having approximately the same overall composition, and/or the same physicochemical properties as the sample matrix.

The term “sample matrix effects” is understood to mean the effect of the sample matrix on the detection of the label or surrogate label in the methods of the present invention, and thus influencing the detection of the analyte.

The term “background sample” is understood to mean the composition used to determine the sample matrix effects and comprises either sample matrix or sample-like matrix. Where the sample itself is used to determine the sample matrix effects, it is referred to as an “original background sample”. As an original background sample has the same composition as the test sample, it also contains the analyte. Alternatively the background sample is a composition comprising sample matrix or sample-like matrix but without analyte, to which analyte has optionally been added.

The term “background-free sample” is understood to mean a composition not comprising sample matrix, sample-like matrix, analyte, or any other compound which is capable of interfering with the detection of the label. Where the influence of specific components of the sample matrix is to be determined, the background-free sample refers to a composition comprising a matrix, similar to the sample matrix, except for these specific components.

The term “correcting for sample matrix effects” is understood to mean (either directly or indirectly) adjusting the values determined by the measurement of the test sample based on the values of the sample matrix effects.

According to a first aspect, the present invention provides an analytical technique for the detection and/or quantification of an analyte in a sample using an analyte-specific probe and a label, whereby the influence of the sample matrix on the detection is determined, allowing correction for the sample matrix effects on the detection.

According to a first embodiment, the method of the invention includes the steps of:
(a) providing the test sample from the sample in which the analyte is to be detected, a background sample comprising sample matrix or sample-like matrix, and a background-free sample, not comprising sample matrix or sample-like matrix
(b) detecting and/or quantifying the analyte in the test sample
(c) detecting the sample matrix effects, by a method comprising the steps of:
(i) contacting the background sample with a predetermined amount of a label,
(ii) contacting the background-free sample with the same predetermined amount of label,
(iii) detecting the label in the background sample and in the background-free sample
(iv) determining the sample matrix effects by determining the difference between the detection of the label in the background sample and in the background-free sample
   (d) correcting the detection and/or quantification of the analyte in the test sample of step (b) with the sample matrix effects determined in (iv).

Optionally the steps (i) to (iii) may be performed with two or more different predetermined amounts of label.

According to a particular embodiment, the background sample used in the detection of the sample matrix effects comprises the sample matrix and more particularly is a fraction of the sample in which the analyte is to be detected and thus its composition is identical to that of the test sample. According to this embodiment, the method comprises the steps of:

(a) providing, from a sample comprising an analyte, both a test sample and a background sample, and further providing a background-free sample;
(b) detecting and/or quantifying the analyte in the test sample
(c) detecting the sample matrix effects, by a method comprising the steps of:
(i) contacting the background sample with a predetermined amount of label
(ii) contacting the background-free sample with the same predetermined amount of label
(iii) detecting the label in the background sample and in the background-free sample
(iv) determining the sample matrix effects by determining the difference between the detection of the label in the background sample and in the background-free sample
   (d) correcting the detection and/or quantification of the analyte in the test sample of step (b) with the sample matrix effects determined in (iv).

Optionally the steps (i) to (iii) may be performed with two or more different predetermined amounts of label.

According to a second aspect, the present invention provides a method for determining sample matrix effects on the detection of a label, the method comprising the steps of:

(a) contacting a predetermined amount of label, a surrogate label or a different label, with a background sample comprising sample matrix or sample-like matrix,
(b) contacting the same predetermined amount of label, surrogate label or different label, with a background-free sample not comprising sample matrix or sample-like matrix or any other compound which is capable of interfering with the detection of the label.
(c) detecting the label, surrogate label or different label, in the background sample and in the background-free sample,
(d) determining the difference between the detection of the label, surrogate label or different label, in the background sample and in the background-free sample, corresponding to the sample matrix effects.

This method provides a measure for the sample matrix effects on the detection of a label in a particular background, which can be used for the correction of the values obtained in the detection and/or quantification of analytes in the samples known or believed to comprise the same matrix.

Optionally the steps (a) to (d) may be performed with two or more different predetermined amounts of label.

Thus, the present invention provides methods and tools, whereby the occurrence of sample matrix effects is determined by detecting a label both in a background sample and a background-free sample, and a system for determining sample matrix effects according to the methods of the present invention. These matrix effects potentially interfere with the detection of an analyte with this label in the sample.

The invention is based on the observation that components of the sample matrix can influence the detection of a label and that this influence can be determined by comparing the detection of that same label, or a label similar thereto in the presence of sample matrix or sample-like matrix and in a background-free sample. The introduction of a label control according to the method of the present invention in a detection method which makes use of a label ensures a more accurate and reliable detection and/or quantification of an analyte in a sample.

The origin of the sample matrix effects determined by the methods of the present invention is variable, and will depend on the nature of the sample. Samples in which detection of an analyte is envisaged according to the present invention include samples from biological material as well as compositions derived or extracted from such biological material. The sample may be any preparation comprising an analyte to be detected. The sample may comprise, for instance, all or a number of components of body tissue or fluid such as but not limited to blood (including plasma and platelet fractions), spinal fluid, mucus, sputum, saliva, semen, stool or urine or any fraction thereof. Exemplary samples can comprise material from whole blood, red blood cells, white blood cells, buffy coat, hair, nails and cuticle material, swabs, including but not limited to buccal swabs, throat swabs, vaginal swabs, urethral swabs, cervical swabs, throat swabs, rectal swabs, lesion swabs, abscess swabs, nasopharyngeal swabs, and the like, lymphatic fluid, amniotic fluid, cerebrospinal fluid, peritoneal effusions, pleural effusions, fluid from cysts, synovial fluid, vitreous humor, aqueous humor, bursa fluid, eye washes, eye aspirates, plasma, serum, pulmonary lavage, lung aspirates, biopsy material of any tissue in the body. The skilled artisan will appreciate that lysates, extracts, or material obtained from any of the above exemplary biological samples are also considered as samples. Tissue culture cells, including explanted material, primary cells, secondary cell lines, and the like, as well as lysates, extracts, supernatants or materials obtained from any cells, tissues or organs, are also within the meaning of the term biological sample as used herein. Samples comprising microorganisms and viruses are also envisaged in the context of analyte detection using the methods of the invention. Materials obtained from forensic settings are also within the intended meaning of the term “sample”. Samples may also comprise foodstuffs and beverages, environmental samples such as water, soil, sand, etc. These lists are not intended to be exhaustive.

In a particular embodiment of the invention, the sample is pre-treated to facilitate the detection of the analyte with the detection method. For instance, typically a pre-treatment of the sample resulting in a semi-purified fraction comprising only those compounds having the same overall nature as the analyte, e.g. extraction of DNA, protein, etc. Methods and kits suitable for the pre-treatment of samples are available in the art.

According to a particular embodiment of the invention, the analyte is a nucleic acid, such as a sequence of genomic DNA or a nucleic acid from a pathogenic microorganism. Typically, in order to detect a genomic DNA in a sample, the sample is heated (e.g. to 100° C.) to ensure denaturation of dsDNA and simultaneously inactivate most enzymatic activity present in the sample. Additionally or alternatively the DNA can be (partially) purified. A variety of methods are available for isolating nucleic acids from samples. Exemplary nucleic acid isolation techniques include (1) organic extraction followed by ethanol precipitation, e.g. using a phenol/chloroform organic reagent (e.g. Ausbel et al., eds., (1995, including supplements through June 2003) Current Protocols in Molecular Biology, John Wiley & Sons, New York), preferably using an automated DNA extractor, e.g. the Model 341 DNA Extractor available from Applied Biosystems (Foster City, Calif.), (2) stationary phase adsorption methods (e.g. Boom et al., U.S. Pat. No. 5,234,809; Walsh et al., BioTechniques 10(4): 506-513 (1991), and (3) salt-induced DNA precipitation methods (e.g. Miller et al., (1988) Nucl. Acids Res., 16(3):9-10), such precipitation methods being typically referred to as “salting-out” methods. Commercially available kits can be used to expedite such methods, for example, Genomic DNA Purification Kit and the Total RNA Isolation System (both available from Promega, Madison, Wis.). Further, such methods have been automated or semi-automated using, for example, the ABI PRISM™ 6700 Automated Nucleic Acid Workstation (Applied Biosystems, Foster City, Calif.) or the ABI PRISM™ 6100 Nucleic Acid PrepStation and associated protocols, e.g. NucPrep™ Chemistry: Isolation of Genomic DNA from Animal and Plant Tissue, Applied Biosystems Protocol 4333959 Rev. A (2002), Isolation of Total RNA from Cultured Cells, Applied Biosystems Protocol 4330254 Rev. A (2002), and ABI PRISM™ Cell Lysis Control Kit, Applied Biosystems Protocol 4316607 Rev. C (2001).

The above pre-treatment methods can further comprise a fragmentation step, e.g. by enzyme digestion, shearing or sonication, and/or an enzymatic amplification step, e.g. by PCR. Most particularly, where sensitive detection of a nucleic acid is envisaged, a PCR amplification of the target DNA can be performed prior to the detection of the analyte. In this context the sample consists of the extracted DNA including the PCR product.

Typical examples of compounds and conditions commonly present in samples or semi-purified fractions of samples, which are capable of causing sample matrix effects include, but are not limited to, ions, large or bulk proteins, bulk DNA, pH, etc. It is however not critical to the present invention that the causative factor of the sample matrix effects be identified.

The method of the present invention can in principle be applied to any analytical detection technique whereby detection of the analyte is performed based on detection of the label in the presence of sample matrix. Most particularly, the invention is of use for analytical detection methods in which the detection of the label is easily affected by factors present in the sample. The method of the present invention is particularly suitable for detection methods based on the detection of label by surface-enhanced resonance Raman spectroscopy (SERRS). In SERRS, use is made of a label which is a SERRS-active substance or dye, which, when illuminating at the resonance frequency of the dye, generates a resonance Raman spectrum. The sensitivity to detect this spectrum is further enhanced by adsorbing the dye onto a roughened metal surface, e.g. nanoparticles of gold, silver, copper and certain other metals (SERRS). A critical factor in SERRS is the efficient adsorption of the dye onto this metal surface. Alternative methods envisage the binding of the dye either directly or indirectly to the metal surface. When the roughened metal surface consists of colloid metal nanoparticles the best signal enhancement is achieved when the colloid nanoparticles are aggregated in a controlled manner. Aggregating agents include acids (e.g. HNO₃ or ascorbic acid), polyamines (e.g. spermine) and inorganic ions (e.g. Cl⁻, I⁻, Na⁺ or Mg²⁺). The presence of such compounds in the sample can thus affect colloid aggregation. Besides the aggregation, components of the sample matrix can also interfere with the adsorption of the dye onto the colloid, thereby negatively affecting the measured SERRS signal.

The methods of the present invention are methods which involve the detection of an analyte. The nature of the analyte to be detected is not critical to the invention and can be any molecule or aggregate of molecules of interest for detection. A non-exhaustive list of analytes includes a protein, polypeptide, peptide, amino acid, nucleic acid, oligonucleotide, nucleotide, nucleoside, carbohydrate, polysaccharide, lipopolysaccharide, glycoprotein, lipoprotein, nucleoproteins, lipid, hormone, steroid, growth factor, cytokine, neurotransmitter, receptor, enzyme, antigen, allergen, antibody, metabolite, cofactor, nutrient, toxin, poison, drug, biowarfare agent, biohazardous agent, infectious agent, prion, vitamin, immunoglobulins, albumin, hemoglobin, coagulation factor, interleukin, interferon, cytokine, a peptide comprising a tumor-specific epitope and an antibody to any of the above substances. An analyte may comprise one or more complex aggregates such as but not limited to a virus, bacterium, microorganism such as Salmonella, Streptococcus, Legionella, E. coli, Giardia, Cryptosporidium, Rickettsia, spore, mold, yeast, algae, amoebae, dinoflagellate, unicellular organism, pathogen or cell, and cell-surface molecules, fragments, portions, components, products, small organic molecules, nucleic acids and oligonucleotides, metabolites of microorganisms.

According to a particular embodiment, an analyte is a DNA such as a gene, viral DNA, bacterial DNA, fungal DNA, mammalian DNA, DNA fragments. The analyte can also be RNA such as viral RNA, mRNA, rRNA. The analyte can also be cDNA, oligonucleotides, or synthetic DNA, RNA, PNA, synthetic oligonucleotides, modified oligonucleotides or other nucleic acid analogue. It may comprise single-stranded and double-stranded nucleic acids. It may, prior to detection, be subjected to manipulations such as digestion with restriction enzymes, copying by means of nucleic acid polymerases, shearing or sonication thus allowing fragmentation to occur.

As indicated above, the present invention provides a label control for detection methods which involve detection by use of a label. Different types of label are envisaged within the context of the present invention, such as, but not limited to, fluorescent, chromogenic or chemiluminescent dye, radio-active, metal and/or magnetic nanoparticles, etc.

Accordingly, the detection steps performed in the methods of the invention will be determined by the label used and include, but are not limited to, fluorescence, colorimetry, absorption, reflection, polarization, refraction, electrochemistry, chemiluminescence, Rayleigh scattering and Raman scattering, SE(R)RS, resonance light scattering, grating-coupled surface plasmon resonance, scintillation counting, magnetic sensors, electrochemical detection (such as anode stripping voltametry), etc.

Suitable labels for use in the different detection methods are numerous and extensively described in the art. Fluorescent labels include but are not limited to fluorescein isothiocyanates (FITC), carboxyfluoresceins such as tetramethylrhodamine (TMR), carboxy tetramethyl-rhodamine (TAMRA), carboxy-X-rhodamine (ROX), sulforhodamine 101 (Texas Red™), Atto dyes (Sigma Aldrich), Fluorescent Red and Fluorescent Orange, phycoerythrin, phycocyanin, and Crypto-Fluor™ dyes, etc. The most common radioisotopes include beta-emitters such as ³H and ¹⁴C, and gamma-emitters, such as iodine-125 (¹²¹I). Other described labels used in quantitative and qualitative assays include but are not limited to dendrimers, quantum dots, up-converting phosphors and nanoparticles.

Where the detection of the analyte in the methods of the invention is based on SE(R)RS, the label is a material which is SE(R)RS-active, i.e. which is capable of generating a SERS or SERRS spectrum when appropriately illuminated, also referred to herein as a SE(R)RS-active label or dye. Non-limiting examples of SE(R)RS-active labels include fluorescein dyes, such as 5-(and 6-) carboxy-4′,5′-dichloro-2′,7′-dimethoxy fluorescein, 5-carboxy-2′,4′,5′,7′-tetrachlorofluorescein and 5-carboxyfluorescein, rhodamine dyes such as 5-(and 6-) carboxy rhodamine, 6-carboxytetramethyl rhodamine and 6-carboxyrhodamine X, phthalocyanines such as methyl, nitrosyl, sulphonyl and amino phthalocyanines, azo dyes, azomethines, cyanines and xanthines such as the methyl, nitro, sulphano and amino derivatives, and succinylfluoresceins.

According to a particular embodiment the SE(R)RS label is a carboxy rhodamine, FAM or TET. It has been demonstrated that a calibration curve for an oligonucleotide labeled with carboxyrhodamine R6G reached a detection limit of 1.05·10⁻¹² M which, taking into account dilution effects, corresponded to a detection of 0.5 femtomoles of the labeled oligonucleotide in the test sample volume. At the same time, the calibration graph of R6G (as well as for FAM and TET) has been shown to be linear over a range from 10⁻⁷ M to 10⁻¹¹ M (LGC “Evaluation of the sensitivity of SERRS-based DNA detection”, January 2004, LGC/Mfb/2004/02, available at http://www.mfbprog.org.uk/themes/theme_publications_item.asp?intThemeID=10&intPublicationID=865).

It is noted that the choice of the label can be influenced by factors such as the resonance frequency of the label, the resonance frequency of other molecules present in the test sample, etc. SE(R)RS-active labels of use for detecting biomolecules are described in the art such as in U.S. Pat. Nos. 5,306,403, 6,002,471, and 6174677.

According to the present invention, the determination of sample matrix effects using a label control is performed independently from (but optionally simultaneously with) the detection of the analyte in the test sample. According to a particular embodiment, the sample matrix effects are determined using the same label as is used for the detection of the analyte in the sample. Alternatively, however, it is envisaged that the label used in the label control is a label which is not the same (i.e. a different label) as the label used in the detection of the analyte. It is envisaged that at least part of the sample matrix effects on the detection of the label will, in most cases, be independent of the nature of the label used. For example, where the sample matrix effects are envisaged in SE(R)RS detection which are due e.g. to effects on the aggregation of the colloids used as SE(R)RS surface, this is expected to have a similar effect independent of the nature of the SE(R)RS dye used. According to a particular embodiment, it is envisaged that the label used in the determination of the sample matrix effects, which is different from the label used in analyte detection, is a label which is comparable in its properties to the label used in the detection of the analyte (similar label).

As indicated above, the provision of a label control according to the present invention is particularly suited for methods wherein the detection of the label is known to be affected by different factors. According to a particular embodiment of the invention, the provision of a label control is applied to an analytical detection method based on surface-enhanced spectroscopies. Detection by surface-enhanced spectroscopies such as surface-enhanced (resonance) Raman spectroscopy (SE(R)RS) is based on the strong enhancement of Raman scattering observed for analytes adsorbed onto a roughened metal surface. Thus, this requires the detection of the label in the presence of an appropriate SE(R)RS-active surface. Typically, the surface is a noble (Au, Ag, Cu) or alkali (Li, Na, K) metal surface. The metal surface may for instance be an etched or otherwise roughened metallic surface, a metal sol or according to a particular embodiment, an aggregation of metal colloid particles as the latter results in enhancements of greater than 108-1014 (Nie and Emory (1997), Science, 275, Kneipp (1999), Chem Rev, 99) of the Raman scattering. The metal nanoparticles making up the SE(R)RS-active surface in the detection methods of the present invention can also be arranged in metal nanoparticle island films, metal-coated nanoparticle-based substrates, polymer films with embedded metal nanoparticles, and the like. The metal surface may be a naked metal or may comprise a metal oxide layer on a metal surface. It may include an organic coating such as of citrate or of a suitable polymer, such as polylysine or polyphenol, to increase its sorptive capacity.

According to a particular embodiment of the invention, the metal colloid particles making up the SE(R)RS-active surface are nanoparticles or colloidal nanoparticles aggregated in a controlled manner such as described in US 2005/0130163 A1. Alternative methods of preparing nanoparticles are known (e.g. U.S. Pat. Nos. 6,054,495, 6,127,120, and 6,149,868). Nanoparticles may also be obtained from commercial sources (e.g. Nanoprobes Inc., Yaphank, N.Y.; Polysciences, Inc., Warrington, Pa.). The metal particles can be of any size as long as they give rise to a SE(R)RS effect. Typically they have a diameter of about 4-50 nm, most particularly between 25-40 nm, depending on the type of metal.

In the detection and/or quantification methods of the present invention making use of SE(R)RS detection methods it is envisaged that the direct or indirect covalent or non-covalent binding or adsorption of the SE(R)RS-active label to the metal surface is directly or indirectly indicative of the presence and/or amount of analyte in the sample. Various options and modes of binding of molecules to SE(R)RS-active surfaces are known in the art and described e.g. in U.S. Pat. No. 6,127,120 and U.S. Pat. No. 6,972,173.

Typically, where the SE(R)RS-active dye is bound to the metal surface through a nucleotide probe, adsorption of the labeled probe to the metal SE(R)RS-active surface is ensured by addition of a monomeric or polymeric polyamine, more particularly a short-chain aliphatic polyamine, such as spermine. Thus, according to one embodiment, the methods of the invention will comprise, prior to detection, addition of a polyamine to the test sample to be detected by SE(R)RS. The polyamine should be introduced at a time which allows its interaction with the analyte and/or the label and/or the labeled analyte-specific probe and/or the labeled surrogate target probe, before the SE(R)RS spectrum is obtained. The polyamine is preferably a short-chain aliphatic polyamine such as spermine, spermidine, 1,4-diaminopiperazine, diethylenetriamine, N-(2-aminoethyl)-1,3-propanediamine, triethylenetetramine and tetraethylenepentamine. Spermine with its four NH₂ groups per repeat unit is particularly suitable for use in the present invention. The polyamine is preferably introduced in the form of an acid salt such as its hydrochloride. It is of most use when the SE(R)RS-active surface is colloidal (vide supra). The amount of polyamine added is preferably of the order of 100 to 1000 times more than would be needed to obtain a monolayer coverage of the surface with the polyamine. Excess polyamine forms a coating on the surface thereby ensuring optimal colloidal aggregation and adsorption of analyte and/or label and/or analyte-specific label.

The addition of a polyamine will ensure an overall increase of DNA binding to the metal surface. Alternatively or additionally, a probe can be modified so as to promote or facilitate chemi-sorption of the probe onto the SE(R)RS-active surface. This can be ensured by at least partially reducing the overall negative charge of analyte-specifc probe. More particularly, where the analyte-specific probe is a nucleotide, this can be ensured by incorporating into the nucleic acid or nucleic acid unit one or more functional groups comprising a Lewis base, such as amino groups, as described in U.S. Pat. No. 6,127,120.

According to a further embodiment, a functional group (such as e.g. a Lewis base) is provided on the SE(R)RS-active label so as to promote or facilitate chemi-sorption onto the SE(R)RS-active surface. Optionally, the SE(R)RS-active label or dye and metal particles are entrapped in a polymer bead as described in US2005/0130163, which can optionally further contain magnetic particles, rendering the beads magnetic which can be of interest in separation (see below).

The present invention provides a label control for detection methods in which sample matrix can interfere with the detection of an analyte. Typically, such methods include methods which do not require the separation of the labeled analyte from either the unbound label and/or other labeled components interfering with the detection and/or quantification of the analyte and/or which do not involve a washing step, separating the analyte from the original sample matrix. According to one embodiment of the invention, the detection of the analyte is ensured based on the specific binding of a label to the analyte, whereby the signal of the label is modified upon binding to the analyte. This can be achieved e.g. by the provision of a molecular beacon e.g. a probe, which is complementary to the target sequence, dually labeled with a dye and a quencher (e.g. Dabcyl) at each of its two ends. In its closed state, the signal of the dye is quenched by the quencher. When the complementary sequence hybridizes to the target DNA, the beacon opens up and a signal can be detected. A further example of labels capable of specifically binding to an analyte and thereby causing a change in signal is provided for SERRS in WO2005/019812. Therein SERRS beacons are described which are dually labeled probes with a different dye at each of its two ends. The second dye is specifically designed such that it is capable of immobilizing the oligonucleotide probe onto an appropriate metal surface. In the absence of target DNA, the beacon is immobilized in the “closed state” on the metal surface, resulting in the detection of a SERRS spectrum corresponding to both dyes. When the complementary sequence hybridizes to the target DNA, the beacon opens up and one of the dyes is removed from the surface. This causes the SERRS signals to change.

According to another embodiment, use is made of fluorophore-labeled nucleotide probes whereby the polarization of the fluorescence of the label increases upon binding to the target nucleic acid (Walker and Linn (1996), Clinical Chemistry, Vol 42, 1604-1608).

According to yet another embodiment of the invention, the provision of a label control is applicable to competitive SE(R)RS methods wherein the detection of the analyte is ensured based on the competitive binding of the analyte and a labeled surrogate target probe to an analyte-specific probe, the latter being associated with a SE(R)RS surface. The labeled surrogate probe is displaced from its binding with the analyte-specific probe as a result of a higher affinity of the analyte-specific probe for the analyte than for the surrogate probe. Such methods ensure an inverse detection of the analyte, as the more analyte present, the more labeled surrogate target probe is displaced from the surface resulting in a decreased SE(R)RS signal.

Typically, the analyte detection of the methods of the present invention involves a labeled analyte-specific probe, which can be a complementary target probe or a surrogate target probe. These probes, intended to either specifically bind to or compete with the analyte (for binding to a second probe), are obtained by linking a compound capable of specifically binding to the analyte or corresponding to at least (a specific) part of the analyte, to a label. The nature of the analyte-specific probe will be determined by the nature of the analyte to be detected. Most commonly, the probe is developed based on a specific interaction with the analyte such as, but not limited to antigen-antibody binding, complementary nucleotide sequences, carbohydrate-lectin, complementary peptide sequences, ligand-receptor, coenzyme-enzyme, enzyme inhibitors-enzyme etc.

According to a particular embodiment of the present invention, the analyte of interest is a nucleotide and the methods of the invention involve the use of at least one labeled analyte-specific probe which is a nucleotide probe, of which the sequence is complementary or similar to at least part of the analyte of interest, most particularly a sequence of the analyte which is specific for the analyte. This nucleotide probe is bound to a label to allow specific detection of the analyte.

Methods for preparing labeled nucleotides and incorporating them into nucleic acids are described in the art (e.g. U.S. Pat. Nos. 4,962,037, 5,405,747, 6,136,543, and 6,210,896).

In a particular embodiment of the invention, a SE(R)RS-active label is used, which is either attached directly to the nucleotide probe or via a linker compound. SE(R)RS-active labels that contain reactive groups designed to covalently react with other molecules, such as nucleotides or nucleic acids, are commercially available (e.g. Molecular Probes, Eugene, Oreg.). SE(R)RS-active labels that are covalently attached to nucleotide precursors may be purchased from standard commercial sources (e.g. Roche Molecular Biochemicals, Indianapolis, Ind.; Promega Corp., Madison, Wis.; Ambion, Inc., Austin, Tex.; Amersham Pharmacia Biotech, Piscataway, N.J.).

According to a particular embodiment of the present invention, detection of the bound labeled probe involves a physical separation of the unbound labeled analyte-specific probe from the labeled analyte-specific probe that is bound to the analyte, within the sample. Separation of the unbound and the bound labeled probe can be achieved by providing a tag to the analyte-specific probe or by providing a secondary analyte-specific separation probe comprising a tag, which can be subjected to physical and/or chemical forces. Typical examples of such tags include magnetic beads (which can be subjected to magnetic forces), glass or polystyrene beads (which can be captured an moved by a light beam; Smith et al. (1995), Science 271:795), or charged groups (which can be subjected to electrokinetic forces; Chou et al. (1999), PNAS 96:11). Where the analyte is a nucleotide sequence, which prior to detection is amplified using PCR, the tag can be incorporated into the PCR product. Tagging of the analyte makes it possible to separate unbound from bound labeled analyte-specific probes to allow individual detection thereof.

Certain aspects of the present invention relate to improved methods for the detection and/or quantification of an analyte, more particularly an analyte in a test sample. While the methods described herein will generally refer to “an analyte” it is equally envisaged that the methods of the present invention can be applied where several analytes are being detected or quantified simultaneously, using different analyte-specific labels. Most particularly, use can be made of different analyte-specific probes which can be differentially detected using the same detection method, such as, but not limited to different fluorescent labels such as, but not limited to, fluorescein isothiocyanates (FITC), carboxyfluoresceins (such as tetramethylrhodamine (TMR), carboxy tetramethyl-rhodamine (TAMRA), carboxy-X-rhodamine (ROX), sulforhodamine 101 (Texas Red™), Atto dyes (Sigma Aldrich), Fluorescent Red and Fluorescent Orange, phycoerythrin, phycocyanin, Crypto-Fluor™ dyes, quantum dots, SE(R)RS-active dyes, and their isotopes. As each of the probes can be made specific for a different analyte, it is possible to measure, for each labeled analyte-specific probe, its detection signal in the test sample. Moreover, it is possible to determine the sample matrix effects for each of the different labels in one label control (based on one background sample and one background-free sample, to which the respective labels have been added) so as to allow compensation for sample matrix effects potentially interfering with the detection of each of the labels.

As indicated above, the methods of the present invention are of particular interest in detection and/or quantification methods based on surface-enhanced (resonance) Raman spectroscopy or SE(R)RS. Though reference is generally made to SE(R)RS herein, it will be understood that detection methods based on other types of surface-enhanced spectroscopies are also envisaged, for example, but not limited to, surface-enhanced fluorescence, normal (Stokes or anti-Stokes) Raman scattering, resonance Raman scattering, coherent (Stokes or anti-Stokes) Raman spectroscopy (CSRS or CARS), Surface-enhanced (resonance) CARS, stimulated Raman scattering, inverse Raman spectroscopy, stimulated gain Raman spectroscopy, hyper-Raman scattering, surface-enhanced hyper-Raman scattering, molecular optical laser examiner (MOLE) or Raman microprobe or Raman microscopy or confocal Raman microspectrometry, three-dimensional or scanning Raman, Raman saturation spectroscopy, time resolved resonance Raman, Raman decoupling spectroscopy or UV-Raman microscopy.

In a particular embodiment of the invention, the detection method of the invention involves SERRS, since operating at the resonant frequency of the label gives increased sensitivity. In this case, the light source used to generate the Raman spectrum is a coherent light source, e.g. a laser, tuned substantially to the maximum absorption frequency of the label being used. This frequency may shift slightly on association of the label with the SE(R)RS-active surface and the analyte and/or analyte binding species, but the skilled person will be well able to tune the light source to accommodate this. The light source may be tuned to a frequency near to the label's absorption maximum, or to a frequency at or near that of a secondary peak in the label's absorption spectrum. SE(R)RS may alternatively involve operating at the resonant frequency of the plasmons on the active surface or (aggregated) colloids.

In the methods of the invention based on SE(R)RS detection, typically one peak, corresponding e.g. to the label's absorption maximum, is selected and excitation is performed only at the wavelength of that peak. Alternatively, where e.g. different analytes are being detected at the same time using different SERRS labels, it may be necessary to detect the entire “fingerprint” spectrum in order to identify each label. In general multivariate analysis methods (such as partial least squares regression, principal components regression, etc,) may be used to perform qualitative and/or quantitative identification of each of the labels present, using either the entire fingerprint spectrum, a spectral range with more than one Raman band, or using one unique Raman band.

Typically, the detection step in a SE(R)RS based detection method will be carried out using incident light from a laser, having a frequency in the visible spectrum. The exact frequency chosen will depend on the label, surface and analyte. Frequencies in the green or red area of the visible spectrum tend, on the whole, to give rise to better surface enhancement effects for noble metal surfaces such as silver and gold. However, it is possible to envisage situations in which other frequencies, for instance in the ultraviolet or the near infrared ranges, might be used. The selection and, if necessary, tuning of an appropriate light source, with an appropriate frequency and power, will be well within the capabilities of one of ordinary skill in the art, particularly on referring to the available SE(R)RS literature.

Excitation sources for use in SE(R)RS-based detection methods include, but are not limited to, nitrogen lasers, helium-cadmium lasers, argon ion lasers, krypton ion lasers, etc. Multiple lasers can provide a wide choice of excitation lines which is critical for resonance Raman spectroscopy. According to a specific embodiment, an argon ion laser is used in a LabRam integrated instrument (Jobin Yvon) at an excitation wavelength of 514.5 nm.

The excitation beam may be spectrally purified with a bandpass filter and may be focused on a substrate using a 6 times objective lens. The objective lens may be used to both excite the sample and to collect the Raman signal, by using a holographic beam splitter to produce a right-angle geometry for the excitation beam and the emitted Raman signal. The intensity of the Raman signals needs to be measured against an intense background from the excitation beam. The background is primarily Rayleigh scattered light and specular reflection, which can be selectively removed with high efficiency optical filters. For example, a holographic notch filter may be used to reduce Rayleigh scattered radiation.

The surface-enhanced Raman emission signal may be detected by a Raman detector. A variety of detection units of potential use in Raman spectroscopy are known in the art and any known Raman detection unit may be used. An example of a Raman detection unit is disclosed e.g. in U.S. Pat. No. 6,002,471. Other types of detectors may be used, such as a charge coupled device (CCD), with a red-enhanced intensified charge-coupled device (RE-ICCD), a silicon photodiode, or photomultiplier tubes arranged either singly or in series for cascade amplification of the signal. Photon counting electronics can be used for sensitive detection. The choice of detector will largely depend on the sensitivity of detection required to carry out a particular assay. Several devices are suitable for collecting SE(R)RS signals, including wavelength selective mirrors, holographic optical elements for scattered light detection and fibre-optic waveguides.

Apparatus for obtaining and/or analyzing a SE(R)RS spectrum may include some form of data processor such as a computer. Once the SE(R)RS signal has been captured by an appropriate detector, its frequency and intensity data will typically be passed to a computer for analysis. Either the fingerprint Raman spectrum will be compared to reference spectra for identification of the detected Raman active compound or the signal intensity at the measured frequencies will be used to calculate the amount of Raman active compound detected.

The present invention provides a method for improving the detection and/or quantification of an analyte in a sample by allowing a correction for sample matrix effects. It will be understood by the skilled person that this can be applied to detection methods in combination with provisions which ensure compensation for optical excitation/collection variations, e.g. using an internal standard such as an isotope-edited label administered to the test sample. An example of such an internal standard is provided in the prior art (Zhang et al. (2005), Anal. Chem. 77(11): 3563-3569).

The present invention provides for improved methods for label-based detection of an analyte. It is envisaged that kits and reagents can be developed which are adapted to the application of the methods of the present invention. According to a further aspect, the present invention provides a system in which the methods described herein can be executed, the system comprising:

(a) means for providing a test sample from the sample in which the analyte is to be detected, a background sample comprising sample matrix or sample-like matrix, and a background-free sample, not comprising sample matrix or sample-like matrix,
(b) means for appropriately contacting the samples with a predetermined amount of label,
(c) means for detecting and/or quantifying the label (in the test sample, the background sample and the background-free sample)
(d) means for determining sample matrix effects by determining the difference between the detection of the label, in the background sample and in the background-free sample, and for correcting the detection and/or quantification of the label in the test sample accordingly.

For example, the present invention includes an integrated device for pathogen detection, e.g. for MRSA detection, meningitis, HIV, bird flu, malaria, etc.

FIG. 3 is a schematic representation of the system 100 according to an embodiment of the present invention. The system 100 is suitable for detecting and optionally quantifying the presence of an analyte in a sample whereby sample matrix effects of the sample on the detection of a label are determined. It comprises a source 101 for providing one or more samples, which can be provided as one or sources such as specialized source 105 of test sample suspected of containing an analyte, source 106 of a background sample comprising sample matrix or sample-like matrix, source 107 of a background-free sample, not comprising sample matrix or sample-like matrix. Additionally the system may comprise a source 102 containing the label, which can be one source or can be provided as a separate source 108 of analyte-specific label (e.g. a labeled analyte-specific probe) and source 109 of label. Optionally, the device comprises at least one additional source 110 of additives serving in the detection. The device further comprises a means 103 wherein the test and background samples are contacted with the respective labels and presented for detection. Optionally this means can be provided as one means 103 or as separate chambers for the contacting of test-sample 111, background sample 112 and background-free sample 113, with the relevant labels and detection. The device further comprises means 104 for:

a) providing sample comprising analyte from source 101 (or 105) and a predetermined amount of the label from source 102 (or 108) to means 103 for contacting the samples with the label or specialized means wherein test sample is contacted with the analyte-specific label 111,
b) providing background sample from source 101 (or 106) and a predetermined amount of label from source 102 (or 109) to means 103 for contacting the samples with the respective label or to specialized means (112) wherein the background sample is contacted with a predetermined amount of label,
c) providing background-free sample from source 101 (or 107) and a predetermined amount of label from source 102 (or 109) to means 103 for contacting the samples with the respective label or to the specialized means 113 wherein the background-free sample is contacted with the same predetermined amount of label.

The means 104 may include gravimetric feeds of the sample and/or analyte and/or background and/or background-free material and may also include an arrangement of pipes/conduits and valves, e.g. selectable and controllable valves, to allow the provision of the fluids from sources 105, 106, 107, 108, 109, and 110 (or from sources 101 and 102) to the contacting means 111, 112, and 113 (or 103). Alternatively, the fluids may be pumped from the sources 105-110 (or 101 and 102) to the contacting means 111-113 (or 103).

According to a particular embodiment, the background sample comprises the sample matrix and more particularly is a fraction of the sample in which the analyte is to be detected and thus its composition is identical to that of the test sample.

The above arrangement of components may be located on a cartridge 117, e.g. a disposable cartridge 117 for use in molecular diagnostics.

Control and analysis circuitry 115, which may be at least partly in the cartridge 117 or may optionally be external to the cartridge 117 and may be provided optionally to control the operation of the means 104. The control and analysis circuitry 115 may be connected to the means 104 by suitable contacts on the surface of the cartridge, e.g. terminals.

Further, means 114 for detecting the label bound to the analyte and also detecting the relevant labels in the background sample and in the background-free sample are provided. Means 114 may be integral with the cartridge 117 or may be external to the cartridge and windows may be provided in the cartridge 117 so that the detection means 114 may detect the sample, etc. The means 114 may be under the control of the control and analysis circuitry 115. The detection means 114 may be a detector able to use one or more or any of the detection methods mentioned above. Signals representative of the detections may be supplied to the control and analysis circuitry 115 which can be adapted to carry out any of the analysis algorithms of the present invention described above. In particular, the control and analysis circuitry 115 may be adapted to determine the sample matrix effects by determining the difference between the detection of the label in the background sample from the detection of the label in the background-free sample and for correcting the detection of the label in the test sample thereby correcting the detection and/or quantification of the analyte in the test sample with the determined sample matrix effects. The results may be displayed on any suitable display means 116 such as a visual display unit, plotter, printer, etc. The control and analysis circuitry 115 may have a connection to a local area or wide area network for transmission of the results to a remote location. Control and analysis circuitry 115 may be implemented in any suitable manner, e.g. dedicated hardware or a suitably programmed computer, microcontroller or embedded processor such as a microprocessor, programmable gate array such as a PAL, PLA or FPGA, or similar.

In accordance with a specific embodiment of the present invention the sample in source 105 may be a solution containing biomolecules such as any of the biomolecules mentioned above and in particular a mixture of DNA molecules. In particular the biomolecules may be DNA obtained from a PCR reaction. This embodiment is particularly useful for use in a molecular diagnostic disposable cartridge which can include a cell lysis station and a PCR reaction station, in particular a multiplexed PCR reaction station. The output of the PCR reaction station then forms the source 105. In this case the label in source 108 will be an analyte-specific oligonucleotide probe to which a label is attached which is suitable for any of the applied detection methods described above. The nucleotide sequence of the probe is chosen such that it can hybridize with the analyte, e.g. is complementary to the sequence of the analyte. A second source 108 may contain a predetermined amount of the same or a different label which is not bound to a nucleotide probe and is therefore not able to bind to the analyte. Source 106 contains a background sample such as a PCR buffer (e.g. Taq PCR buffer, 50 mM KCl, 10 mM Tris HCl, 1.5 mM MgCl₂, 0.1% gelatin). Source 107 contains a background-free sample such as water or a buffer solution suitable for undisturbed detection of the label. When a surface-enhanced spectroscopy method is used for detection of the label, an additional source 110 may contain a suitable metal surface such as colloid particles or beads coated with metal surface, especially aggregatable colloid particles or beads coated with metal surface. A second additional source 110 may contain an aggregating agent such as spermine.

In this specific embodiment, appropriately providing and contacting of reagents from the sources 105 to 110 may result in chamber 111 containing PCR reaction output and a predetermined amount of labeled oligonucleotide probe, chamber 112 containing PCR reaction output and a predetermined amount of label, chamber 113 containing water and a predetermined amount of label, and in addition all three chambers 111-113 containing aggregated colloid particles for enabling surface-enhanced spectroscopic detection by the detection means 114.

It is to be understood that although preferred embodiments, specific constructions and configurations, as well as materials, have been discussed herein for devices according to the present invention, various changes or modifications in form and detail may be made without departing from the scope and spirit of this invention.

### EXAMPLE

### Example 1

### Determination of the Effect of Buffer Composition on SERRS Detection of a SERRS-Active Label

A synthetic oligonucleotide (19 bp, TGCTTCTACACAGTCTCCT) labeled at its 5′ end with rhodamine-6G was used to study the effect of buffer composition on the detected SERRS intensity. The oligonucleotide was dissolved in a background-free sample, i.e. water, at a concentration of 2·10⁻⁹ M. To investigate the effect of buffer composition on SERRS measurement, the same amount of oligonucleotide was dissolved in a background sample, i.e. water containing 10 mM NaCl. For SERRS measurements 10 μl of each sample was mixed with 10 μl spermine tetrachloride (100 mM in water, freshly prepared). To these solutions 250 μl water and 250 μl silver nanoparticles (prepared as described by Munro et al. (1995), Langmuir, 11:3712-3720) were added. Immediately after mixing SERRS spectra were taken from both samples using a LabRam system (Jobin Yvon) with an Argon laser providing excitation at 514.5 nm. A comparison of the two spectra showed a significant difference in SERRS intensity. This was most likely caused by an increase in the amount of aggregates and/or a change of the aggregate size by the addition of NaCl.

### Example 2

### Detection and Quantification of a Specific Gene in a Sample Using Competitive SERRS and Correction for Sample Matrix Effects

Highly pathogenic avian influenza caused by certain subtypes of influenza A virus in animal populations, particularly chickens, poses a continuing global human public health risk. Direct human infection by the avian influenza A subtype H5N1 virus has been responsible for considerable human mortality recently, stressing the need for rapid and accurate diagnosis. Type A influenza viruses are subtyped on the basis of antigenic differences in the external glycoproteins, the hemagglutinins (HA) and the neuraminidases (NA). The present invention offers a novel, rapid and accurate approach to viral subtyping by using RT-PCR and SERRS of viral nucleic acids.

Viral RNA is extracted from clinical samples and cDNA complementary for viral RNA is generated using viral reverse transcriptase and random primers according to Wright et al. (1995), J. Clin. Microbiol., 33:1180-1184. Multiplex PCR is carried out with two sets of primers specific for the HA and NA genes of influenza virus subtype H5N1, as described in “Recommended laboratory tests to identify avian influenza A virus in specimens from humans”, WHO Geneva, June 2005), designed to yield PCR products of 219 and 616 bp, respectively. Amplified products are subsequently detected by competitive SERRS. Therefore, two analyte-specific probes are designed to be complementary to a region within the HA and NA genes, respectively, and having the surface-seeking group propargylamine for attachment to a silver nanoparticle. Two additional synthetic oligonucleotides, so-called surrogate target probes, labeled with the SERRS dyes HEX and TET, respectively, are designed to be complementary to a portion of the HA- and NA-specific probes, respectively, except for one mismatch (SNP).

Two label solutions are prepared. The first label solution contains predetermined amounts of HA- and NA-specific probes and corresponding surrogate target probes for detection of amplified H5N1 viral HA and NA genes. Due to the presence of the SNP, the HA- and NA-specific probes and their corresponding surrogate target probes are loosely annealed in the first label solution. The second label solution, prepared in duplicate, solely contains a predetermined amount of the SERRS dyes HEX and TET.

To determine a reference point for the SERRS measurements of HEX and TET in the various samples, 10 μl of each label solution is mixed with 10 μl spermine tetrachloride (100 mM in water, freshly prepared). To these solutions 250 μl water and 250 μl silver nanoparticles (prepared as described by Munro et al. (1995), Langmuir, 11:3712-3720) are added. Immediately after mixing SERRS spectra are taken from the prepared solutions using a LabRam system (Jobin Yvon) with an Argon laser providing excitation at 514.5 nm.

The output of the PCR reaction is then split into two equal portions to provide a test sample and a background sample. Water is provided as a background-free sample. For detection of amplified H5N1 viral HA and NA genes the test sample is added to the first label solution containing predetermined amounts of HA- and NA-specific probes, surrogate target probes and aggregated silver colloids. For determination of sample matrix effects the background and the background-free samples are each added to a second label solution, containing a predetermined amount of SERRS dye and aggregated silver colloids.

Incubation at an appropriate temperature allows for the analyte DNA's in the test sample to compete with the surrogate target probes for hybridization to the HA- and NA-specific probes, the latter having attached to the aggregated silver colloids. Since the surrogate target probes are not perfectly complementary to the HA- and NA-specific probes, hybridization of the analyte DNA's to the HA- and NA-specific probes is more stable and the surrogate target probes are displaced from the metal surface resulting in a decrease in SERRS intensity of the HEX and TET dyes.

The background and the background-free samples are also incubated and their SERRS spectra compared to quantify the sample matrix effects generated by PCR buffer compounds, residual DNA's, etc.

By compensating for sample matrix effects an accurate detection of amplified viral HA and NA genes is thus achieved using competitive SERRS.

1. A method for determining sample matrix effects of a sample on the detection of a label, the method comprising the steps of:
(a) contacting a predetermined amount of said label or a different label, with a background sample comprising sample matrix or sample-like matrix,
(b) contacting a predetermined amount of said label, or of said different label, with a background-free sample not comprising sample matrix or sample-like matrix or any other compound which is capable of interfering with the detection of the label,
(c) detecting said label or said different label in said background sample and said background-free sample, and
(d) determining a difference between the detection of said label or said different label in said background sample and in said background-free sample, corresponding to said sample matrix effects.

2. A method for determining sample matrix effects on the detection of an analyte in a sample, the method comprising the steps of:
(a) providing a test sample from said sample in which said analyte is to be detected, a background sample comprising sample matrix or sample-like matrix, and a background-free sample, not comprising sample matrix or sample-like matrix,
(b) detecting and/or quantifying the analyte in said test sample using a label,
(c) detecting said sample matrix effects, by a method comprising the steps of:
(i) contacting said background sample with a predetermined amount of said label or a different label,
(ii) contacting said background-free sample with a predetermined amount of label or said different label,
(iii) detecting said label or said different label in said background sample and in said background-free sample,
(iv) determining said sample matrix effects by determining a difference between the detection of said label or said different label in said background sample and said background-free sample,
(d) correcting the detection and/or quantification of said analyte in said test sample of step (b) with said sample matrix effects determined in (iv).

3. The method of claim 2, wherein said background sample is a fraction of said sample in which the analyte is to be detected.

4. The method of claim 2, wherein said background sample comprises sample matrix or sample-like matrix.

5. The method according to claim 1, wherein the correction for sample matrix effects is performed using two or more predetermined amounts of label.

6. The method according to claim 1, wherein said analyte is selected from the group consisting of a nucleic acid, a protein, a carbohydrate, a lipid, a chemical substance, an antibody, a microorganism, and a eukaryotic cell.

7. The method according to claim 1, wherein said detection in step (c) or in steps (b) and (c), respectively, is performed using an optical detection method.

8. The method according to claim 7, wherein said optical detection method is SE(R)RS and wherein said label or said different label is a SE(R)RS-active labels.

9. The method according to claim 8, further comprising, prior to detection, contacting of said label or said different label with a SE(R)RS-active surface.

10. The method according to claim 9, wherein said SE(R)RS-active surface is a colloidal suspension of silver or gold nanoparticles, or aggregated colloids thereof.

11. The method according to claim 2, wherein said detection of said analyte is performed using a labeled analyte-specific probe.

12. The method according to claim 9, wherein said analyte-specific probe is provided with a binding-sensitive label.

13. The method according to claim 9, wherein said analyte is a nucleotide sequence and said labeled analyte-specific probe is a nucleotide or nucleotide analogue sequence having a sequence complementary to a sequence within said analyte.

14. The method according to claim 2, wherein said detection of said analyte is performed using an analyte specific probe capable of binding to a SE(R)RS-active surface and a labeled surrogate probe, and whereby said analyte competes with said labeled surrogate probe for the binding to said analyte-specific probe.

15. The method according to claim 2, wherein said detection and/or quantification of said analyte in step (b) is based on the detection of a labeled analyte-specific probe or labeled surrogate probe and wherein said correction step (d) is performed by correcting said detection of said labeled analyte-specific probe or labeled surrogate probe with said sample matrix effects determined in (iv).

16. A system for determining sample matrix effects of a sample on the detection of a label, comprising:
(a) means for contacting a predetermined amount of said label or a different label, with a background sample comprising sample matrix or sample-like matrix,
(b) means for contacting a predetermined amount of said label, or of said different label, with a background-free sample not comprising sample matrix or sample-like matrix or any other compound which is capable of interfering with the detection of the label,
(c) means for detecting said label or said different label in said background sample and said background-free sample, and
(d) means for determining a difference between the detection of said label or said different label in said background sample and in said background-free sample, corresponding to said sample matrix effects.

17. A system for determining sample matrix effects on the detection of an analyte in a sample comprising:
(a) means for providing a test sample from said sample in which said analyte is to be detected, a background sample comprising sample matrix or sample-like matrix, and a background-free sample, not comprising sample matrix or sample-like matrix,
(b) means for detecting and/or quantifying the analyte in said test sample using a label,
(c) means for contacting said background sample with a predetermined amount of said label or a different label,
(d) means for contacting said background-free sample with a predetermined amount of said label or said different label,
(e) means for detecting said label or said different label in said background sample and in said background-free sample,
(f) means for determining said sample matrix effects by determining a difference between the detection of said label or said different label in said background sample and said background-free sample,
(g) means for correcting the detection and/or quantification of said analyte in said test sample responsive to the means for determining the sample matrix effects.

18. The system according to claim 16, further comprising a first source (101) of one or more samples selected from the group consisting of the test sample containing said analyte, background sample and background-free sample, and a second source (102) of one or more labels and optionally a third source of additives (110).

19. The system according to claim 18, wherein said first source (101) comprises specialized chambers for said test sample containing said analyte, background sample and background-free sample, respectively.

20. The system according to claim 16, further comprising chambers for contacting said test sample containing said analyte, background sample and background-free sample with said labels.

21. The system of claim 18, wherein said second source (102) of said one or more labels, comprises a chamber (108) for an analyte-specific label and a chamber (109) for a label.

22. A disposable cartridge (117) for use in a system for determining sample matrix effects on the detection of an analyte in a sample, comprising:
a first source (101) of one or more samples selected from the group consisting of the test sample containing said analyte, background sample and background-free sample, and
a second source (102) of one or more labels and optionally a third source of additives (110), and
means for contacting said background sample with a predetermined amount of said label or a different label, for contacting said background-free sample with a predetermined amount of said label or a different label, and for contacting the test sample with a predetermined amount of said label or a different label, and
a window to allow detection of said label or said different label in said test sample, said background sample and said background-free sample.
